(19) United States
(12) Patent Application Publication
(10) Pub. No.: US 2025/0032086 A1
(43) Pub. Date: Jan. 30, 2025

(54) METHOD AND SYSTEM FOR ENHANCED VISUALIZATION OF FIBROIDS WITH RESPECT TO A UTERUS AND ENDOMETRIUM IN ULTRASOUND IMAGING

(71) Applicant: GE Precision Healthcare LLC, Waukesha, WI (US)

(72) Inventors: Stephan Anzengruber, Ried im Innkreis (AT); Balint Czupi, Seewalchen am Attersee (AT); Pavan Kumar V. Annangi, Bangalore, Karnataka (IN); Deepa Anand, Bangalore (IN); Bhushan Patil, Pune (IN); Cindy L. Smrt, Carrollton, TX (US); Martin Swoboda, Hohenzell (AT)

(21) Appl. No.: 18/225,811

(22) Filed: Jul. 25, 2023

(51) Int. Cl. A61B 8/08; A61B 8/00; G06T 7/00; G06T 7/12

(52) U.S. Cl. CPC A61B 8/085; A61B 8/469; A61B 8/488; G06T 7/0012; G06T 7/12; G06T 2207/10132; G06T 2207/30004

(57) ABSTRACT

Systems and methods for enhancing visualization and documentation of fibroid quantity, size, and location with respect to a uterus and endometrium in ultrasound imaging are provided. The method includes receiving, by at least one processor, an ultrasound volume including ultrasound image data of a region of interest that includes a uterus. The method includes automatically segmenting, by the at least one processor, the uterus and an endometrium in the ultrasound volume. The method includes segmenting one or more fibroids in the ultrasound image data. The method includes generating a classification of each of the one or more fibroids based on a location of each of the one or more fibroids with respect to the endometrium and the uterus. The method includes causing, by the at least one processor, a display system to present at least one image identifying the segmented uterus, endometrium, and one or more fibroids.

### FIELD

Certain embodiments relate to ultrasound imaging. More specifically, certain embodiments relate to a method and system for enhancing visualization and documentation of fibroid quantity, size, and location with respect to a uterus and endometrium in ultrasound imaging.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Fibroids are non-cancerous growths in a uterus that are common in women of all ages. Fibroids can create several gynecological and obstetric issues, such as infertility. Fibroids can occur in clusters and may cause physical discomfort as the fibroids grow in size. The spatial location of fibroids with respect to an endometrium and uterine serosa plays a critical role in determining a severity and a treatment pathway. For example, fibroids may need to be surgically treated if occurring in close proximity with the endometrium such that the fibroids compress the endometrium. In the case of surgical treatment, the quantity, size, and location of the fibroids with respect to the uterus and endometrium are important for surgical planning purposes. The quantity, size, and location of fibroids are also important for monitoring changes in fibroids over time (i.e., longitudinal analysis).

Although performing a 3D ultrasound scan ensures a 3D context is preserved, drawing 3D contours for each fibroid is tedious with a high skill barrier. Moreover, manual contouring of the uterus and endometrium is extremely time consuming, resulting in reduced productivity. Consequently, the documentation of fibroid quantity, size, and location for surgical planning purposes and/or fibroid monitoring over time is often performed by hand drawings with pen and paper, or using magnetic resonance imaging (MRI) if available. However, MRI is expensive to perform and not always available. Furthermore, the accuracy of hand drawings with pen and paper is limited, particularly with respect to fibroid size and specific location.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for enhancing visualization and documentation of fibroids with respect to a uterus and endometrium in ultrasound imaging, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to enhance visualization and documentation of fibroids with respect to a uterus and endometrium in ultrasound imaging, in accordance with various embodiments.

FIG. 2 is a block diagram of an exemplary medical workstation that is operable to enhance visualization and documentation of fibroids with respect to a uterus and endometrium in ultrasound imaging, in accordance with various embodiments.

FIG. 3 is a screenshot of an exemplary display comprising a mid-sagittal ultrasound plane of a uterus, in accordance with various embodiments.

FIG. 4 is a screenshot of an exemplary display comprising a mid-sagittal ultrasound plane of a uterus having a uterine trace through an endometrium, in accordance with various embodiments.

FIG. 5 is a screenshot of an exemplary display comprising an A-plane, B-plane, C-plane, and volume rendering of a uterus and endometrium segmented in an ultrasound volume, in accordance with various embodiments.

FIG. 6 is a screenshot of an exemplary display comprising an A-plane, B-plane, C-plane, and volume rendering of a uterus, endometrium, and fibroids segmented in an ultrasound volume, in accordance with various embodiments.

FIG. 7 is a screenshot of an exemplary display updated from the exemplary display of FIG. 6 in response to a parallel shift operation to scroll between ultrasound image slices extending from a first end to a second end of an ultrasound volume, the exemplary updated display comprising an A-plane, B-plane, C-plane, and volume rendering of a uterus, endometrium, and fibroids segmented in an ultrasound volume, in accordance with various embodiments.

FIG. 8 is a flow chart illustrating exemplary steps that may be utilized for enhancing visualization and documentation of fibroids with respect to a uterus and endometrium in ultrasound imaging, in accordance with various embodiments.

### DETAILED DESCRIPTION

Certain embodiments may be found in a method and system for enhancing visualization and documentation of fibroid quantity, size, and location with respect to a uterus and endometrium in ultrasound imaging. Aspects of the present disclosure have the technical effect of acquiring an ultrasound volume of a uterus based on a two-dimensional (2D) ultrasound acquisition of a mid-sagittal plane of a uterus. Various embodiments have the technical effect of automatically segmenting a uterus and endometrium depicted in an ultrasound volume. Certain embodiments have the technical effect of enabling scrolling through an ultrasound volume of a uterus from end-to-end to separately display parallel A-plane ultrasound slices. Aspects of the present disclosure have the technical effect of providing automated fibroid segmentation in ultrasound volumes based on color Doppler ultrasound data, semi-automated fibroid segmentation in ultrasound volumes based on user-selected seed points, and/or manual fibroid segmentation based on manual traces of fibroids. Various embodiments have the technical effect of classifying and/or prompting a classification from a user of each segmented fibroid with respect to the uterus and endometrium. Certain embodiments have the technical effect of presenting a volume rendering of the segmented uterus, endometrium, and fibroids. Aspects of the present disclosure have the technical effect of automatically performing volumetric and/or estimated diameter measurements of the uterus, endometrium, and/or each of the fibroids. Various embodiments have the technical effect of facilitating longitudinal analysis of fibroid quantity, size, and location with respect to a uterus and endometrium in ultrasound imaging. Certain embodiments have the technical effect of enhancing surgical planning and/or fibroid monitoring over time via ultrasound imaging. Aspects of the present disclosure have the technical effect of generating and storing reports comprising ultrasound image data and/or volume renderings of the endometrium, uterus, and fibroids, fibroid classifications, and measurements.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment.” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging. Shear Wave Elasticity Imaging (SWEI), Strain Elastography, Tissue Velocity Imaging (TVI), Power Doppler Imaging (PDI), B-flow, Micro Vascular Imaging (MVI), Ultrasound-Guided Attenuation Parameter (UGAP), and the like.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

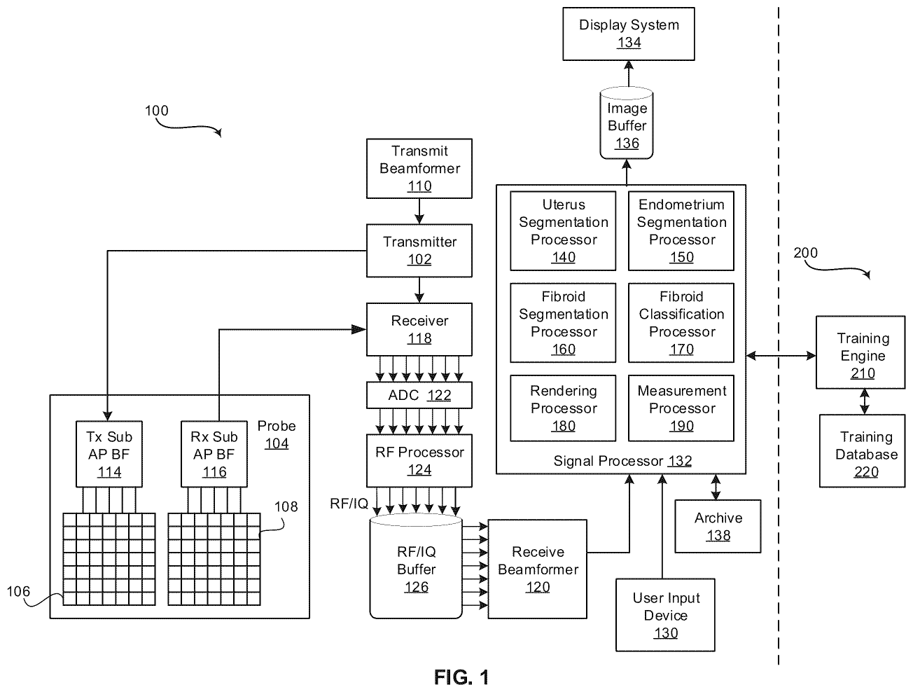

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to enhance visualization and documentation of fibroids with respect to a uterus and endometrium in ultrasound imaging, in accordance with various embodiments. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two-dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as a uterus or any suitable anatomical structure. In a representative embodiment, the ultrasound probe 104 may be a transvaginal probe, a transabdominal probe, and/or any suitable ultrasound probe.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or a plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, image acquisition and scan parameters, settings, configuration parameters, select protocols and/or templates, change scan mode, select seed points in ultrasound images corresponding to fibroids, perform manual traces of fibroids in ultrasound images, select a probe orientation, provide fibroid classifications based on a location of a fibroid with respect to a uterus and endometrium, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134. and/or the archive 138. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, graphic processing units, microprocessors, microcontrollers, and/or the like. The signal processor 132 may be an integrated component, or may be distributed across various locations, for example. In an exemplary embodiment, the signal processor 132 may comprise a uterus segmentation processor 140, an endometrium segmentation processor 150, a fibroid segmentation processor 160, a fibroid classification processor 170, a rendering processor 180, and a measurement processor 190, and may be capable of receiving input information from a user input device 130 and/or archive 138, generating an output displayable by a display system 134, and manipulating the output in response to input information from a user input device 130, among other things. The signal processor 132, uterus segmentation processor 140, endometrium segmentation processor 150, fibroid segmentation processor 160, fibroid classification processor 170, rendering processor 180, and measurement processor 190 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

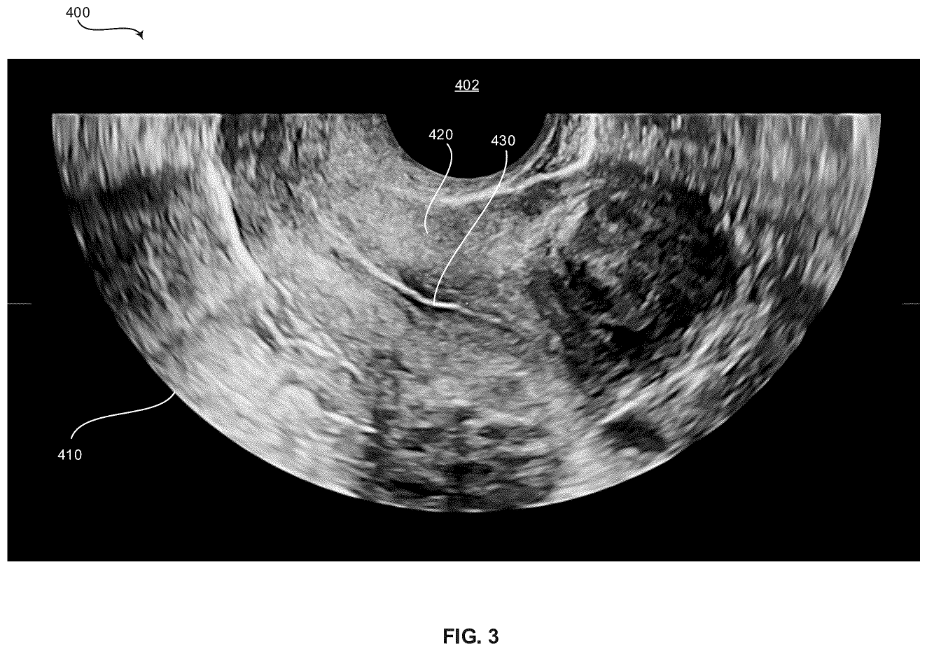

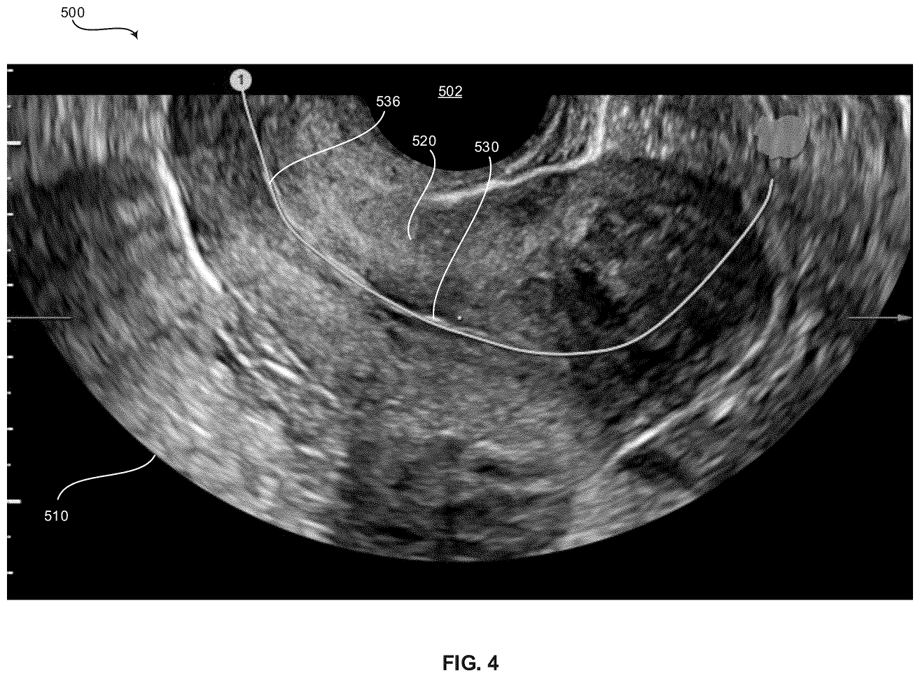

In various embodiments, the ultrasound probe 104 of the ultrasound system 100 may be manipulated by an ultrasound operator to identify a right endometrial plane via two-dimensional (2D) ultrasound scanning prior to initiating a three-dimensional (3D) ultrasound image acquisition. The identification of the right endometrial plane by 2D scanning ensures the mid-sagittal plane of the 3D acquisition is aligned with the endometrium of the uterus. FIG. 3 is a screenshot 400 of an exemplary display 402 comprising a mid-sagittal ultrasound plane 410 of a uterus 420, in accordance with various embodiments. Referring to FIG. 3, the screenshot 400 illustrates a display 402 of a mid-sagittal ultrasound plane 410 depicting a uterus 420 and endometrium 430. In an exemplary embodiment, the ultrasound operator may optionally perform a uterine trace through the endometrium 430 in the right endometrial plane (i.e., mid-sagittal plane) 410 of the 2D image prior to initiating the 3D ultrasound acquisition. FIG. 4 is a screenshot 500 of an exemplary display 502 comprising a mid-sagittal ultrasound plane 510 of a uterus 520 having a uterine trace 536 through an endometrium 530, in accordance with various embodiments. The uterine trace 536 may be provided by the ultrasound operator via the user input deice 130, such as a mousing device or a touchscreen display. For example, the ultrasound operator may manipulate a mousing device to drag a pointer across the display device 134 through the endometrium 530 to mark the uterine trace 536. As another example, the ultrasound operator may drag a finger or stylus across the display device 134 through the endometrium 530 to mark the uterine trace 536. The uterine trace 536 allows the ultrasound operator to confirm the mid-sagittal plane 510 has been acquired prior to switching to the 3D image acquisition. In certain embodiment, the uterine trace 536 may be provided to the uterus segmentation processor 140 and/or endometrium segmentation processor 150 to assist with segmentation of the uterus 420, 520 and/or endometrium 430, 530. For example, the uterine trace 536 may be used by the uterus segmentation processor 140 and/or endometrium segmentation processor 150 to test the alignment of the segmented output. As another example, the endometrium segmentation processor 150 may use the uterine trace 536 to complete the cervical canal as an extension to the endometrium segmentation. In an exemplary embodiment, the uterine trace 536 may not be performed.

The signal processor 132 may include a uterus segmentation processor 140 that comprises suitable logic, circuitry, interfaces and/or code that may be operable to receive a three-dimensional (3D) ultrasound volume image. The uterus segmentation processor 140 may be configured to automatically segment a uterus 420, 520 from the ultrasound volume image. The ultrasound volume image may be a gynecological uterine ultrasound scan, among other things. For example, the uterus segmentation processor 140 may include image analysis algorithms, artificial intelligence algorithms, one or more deep neural networks (e.g., a convolutional neural network) and/or may utilize any suitable form of image analysis techniques or machine learning processing functionality configured to automatically segment a uterus 420, 520 from an ultrasound volume image (i.e., 3D). In various embodiments, the uterus segmentation processor 140 may be provided as a deep neural network that may be made up of, for example, an input layer, an output layer, and one or more hidden layers in between the input and output layers. Each of the layers may be made up of a plurality of processing nodes that may be referred to as neurons. For example, the uterus segmentation processor 140 may include an input layer having a neuron for each voxel or a group of voxels from an ultrasound volume image, such as a gynecological uterine ultrasound scan. The output layer may have neurons corresponding to a segmented uterus. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the uterus segmentation processor 140 deep neural network (e.g., convolutional neural network) may segment a uterus 420, 520 depicted in an ultrasound volume image with a high degree of probability. The uterus segmentation processor 140 may provide the ultrasound volume having the segmented uterus to the endometrium segmentation processor 150 and/or may store the ultrasound volume having the segmented uterus at archive 138 and/or any suitable data storage medium.

The signal processor 132 may include an endometrium segmentation processor 150 that comprises suitable logic, circuitry, interfaces and/or code that may be operable to receive the three-dimensional (3D) ultrasound volume image having the segmented uterus. The endometrium segmentation processor 150 may be configured to automatically segment an endometrium 430, 530 from the ultrasound volume image having the segmented uterus. For example, the endometrium segmentation processor 150 may include image analysis algorithms, artificial intelligence algorithms, one or more deep neural networks (e.g., a convolutional neural network) and/or may utilize any suitable form of image analysis techniques or machine learning processing functionality configured to automatically segment an endometrium 430, 530 from the ultrasound volume image (i.e., 3D). In various embodiments, the endometrium segmentation processor 150 may be provided as a deep neural network that may be made up of, for example, an input layer, an output layer, and one or more hidden layers in between the input and output layers. Each of the layers may be made up of a plurality of processing nodes that may be referred to as neurons. For example, the endometrium segmentation processor 150 may include an input layer having a neuron for each voxel or a group of voxels from an ultrasound volume image. The output layer may have neurons corresponding to a segmented endometrium. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the endometrium segmentation processor 150 deep neural network (e.g., convolutional neural network) may segment an endometrium 430, 530 depicted in an ultrasound volume image with a high degree of probability. The endometrium segmentation processor 150 may provide the ultrasound volume having the segmented uterus and endometrium to the rendering processor 180 for presenting ultrasound images and a volume rendering at the display system 134 as described below and/or may store the ultrasound volume having the segmented uterus and endometrium at archive 138 and/or any suitable data storage medium.

The signal processor 132 may include a rendering processor 180 that comprises suitable logic, circuitry, interfaces and/or code that may be operable to receive the ultrasound volume having the segmented uterus and endometrium from the uterus segmentation processor 140 and/or endometrium segmentation processor 150, or retrieve from archive 138 and/or any suitable data storage medium, the ultrasound volume having the segmented uterus and endometrium. The rendering processor 180 may be configured to generate one or more renderings from the segmented ultrasound volume. For example, the rendering processor 180 may comprise suitable logic, circuitry, interfaces and/or code that may be operable to generate volume renderings, cross-sectional images, an A-plane (i.e., the plane parallel to the acquisition plane), a B-plane (i.e., the plane perpendicular to the A-plane but still parallel to the ultrasound beam), a C-plane (i.e., often referred to as the coronal plane, or the two-dimensional slices at various depths from and parallel to the transducer face, and perpendicular to the ultrasound beam), curved slices (i.e., a 2D image slice extracted from a 3D volume along a curved anatomical structure, such as an endometrium of a uterus), and/or any suitable ultrasound images. The rendering processor 180 may be configured to cause a display system 134 to present the generated image(s) and/or store the generated image(s) at archive 138 and/or any suitable data storage medium.

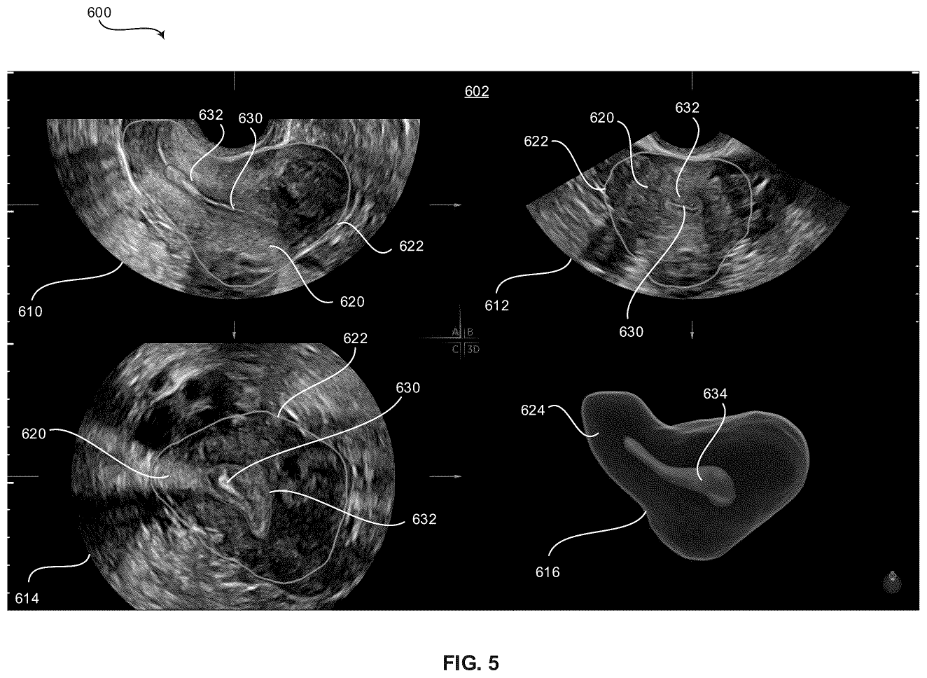

FIG. 5 is a screenshot 600 of an exemplary display 602 comprising an A-plane 610, B-plane 612, C-plane 614, and volume rendering 616 of a uterus 620, 622, 624 and endometrium 630, 632, 634 segmented in an ultrasound volume, in accordance with various embodiments. Referring to FIG. 5, the screenshot 600 shows a display 602 having an A-plane 610, B-plane 612, C-plane 614, and volume rendering 616. The A-plane 610 may correspond with the mid-sagittal plane of the volume ultrasound image of the uterus 620. The B-plane 612 may correspond with the transverse plane of the volume ultrasound image of the uterus 620. The C-plane 614 may correspond with the coronal plane of the volume ultrasound image of the uterus 620. The volume rendering 616 is a two-dimensional (2D) projection of the three-dimensional (3D) ultrasound volume and comprises a rendering of the uterus 624 and endometrium 634. The A-plane 610, B-plane 612, and C-plane 614 are ultrasound image slices extracted from the acquired 3D ultrasound volume. The A-plane 610, B-plane 612, and C-plane 614 depict the uterus 620 and endometrium 630. The A-plane 610, B-plane 612, and C-plane 614 may comprise markings 622, 632, colorized pixels, or the like identifying the segmentation of the uterus 622 and the segmentation of the endometrium 632. In various embodiments, the 3D ultrasound volume image comprises a plurality of parallel A-plane ultrasound image slices 610 spanning from one end of the 3D ultrasound volume to the opposite end of the 3D ultrasound volume. The ultrasound operator or other medical professional may operate the user input device 130 to scroll through the parallel A-plane ultrasound image slices 610 of the 3D ultrasound volume.

The signal processor 132 may include a fibroid segmentation processor 160 that comprises suitable logic, circuitry, interfaces and/or code that may be operable to automatically, semi-automatically, and/or manually segment fibroids in the 3D ultrasound volume image and/or 2D ultrasound images slices 610, 612, 614 extracted from the 3D ultrasound volume image. The fibroid segmentation processor 160 may be configured to manually segment fibroids from the 3D ultrasound volume image and/or 2D ultrasound images slices 610, 612, 614 extracted from the 3D ultrasound volume image. For example, the fibroid segmentation processor 160 may be configured to receive manual traces provided by the ultrasound operator or other medical professional via a user input device 130. As an example, the ultrasound operator or other medical professional may scroll through the parallel A-plane ultrasound image slices 610 and manually trace the contours of identified fibroids using a mousing device, touchscreen, and/or any suitable user input device 130. As another example, the manual segmentation of the fibroids may be performed using an ellipse tool. As another example, the manual segmentation of the fibroids may be performed using one, two, or three orthogonal caliper measurements.

The fibroid segmentation processor 160 may additionally and/or alternatively be configured to automatically segment fibroids from the 3D ultrasound volume image and/or 2D ultrasound images slices 610, 612, 614 extracted from the 3D ultrasound volume image with the assistance of color Doppler ultrasound data. For example, fibroids may be difficult to distinguish from adenomyosis, polyps, and other structures. Accordingly, the ultrasound system 100 may be operable to perform a color Doppler acquisition. The color Doppler ultrasound data provides blood flow characteristics allowing fibroids to be distinguished from other structures, such as adenomyosis and polyps, for example. As another example, contrast agents may be used to visually enhance fibroids for automatic segmentation. The fibroid segmentation processor 160 may include image analysis algorithms, artificial intelligence algorithms, one or more deep neural networks (e.g., a convolutional neural network) and/or may utilize any suitable form of image analysis techniques or machine learning processing functionality configured to automatically segment fibroids from the ultrasound volume image and/or 2D ultrasound image slices 610, 612, 614 extracted from the 3D volume image using the color Doppler ultrasound image data. In various embodiments, the fibroid segmentation processor 160 may be provided as a deep neural network that may be made up of, for example, an input layer, an output layer, and one or more hidden layers in between the input and output layers. Each of the layers may be made up of a plurality of processing nodes that may be referred to as neurons. For example, the fibroid segmentation processor 160 may include an input layer having a neuron for each pixel, voxel, group of pixels, or group of voxels from the ultrasound image data. The output layer may have neurons corresponding to segmented fibroid(s). Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The neurons of a fourth layer may learn blood flow characteristics of the detected shapes. The processing performed by the fibroid segmentation processor 160 deep neural network (e.g., convolutional neural network) may segment fibroids depicted in ultrasound image data with a high degree of probability. The fibroid segmentation processor 160 may provide the ultrasound volume having the segmented uterus 620, 622, endometrium 630, 632, and fibroids to the rendering processor 180 for presenting ultrasound images and a volume rendering at the display system 134 and/or may store the ultrasound volume having the segmented fibroids, uterus 620, 622, and endometrium 630, 632 at archive 138 and/or any suitable data storage medium.

The fibroid segmentation processor 160 may additionally and/or alternatively be configured to semi-automatically segment fibroids from the 3D ultrasound volume image and/or 2D ultrasound images slices 610, 612, 614 extracted from the 3D ultrasound volume image with the assistance of user provided seed points identifying the fibroids. For example, the fibroid segmentation processor 160 may include image analysis algorithms, artificial intelligence algorithms, one or more deep neural networks (e.g., a convolutional neural network) and/or may utilize any suitable form of image analysis techniques or machine learning processing functionality configured to semi-automatically segment fibroids from the ultrasound volume image and/or 2D ultrasound image slices 610, 612, 614 extracted from the 3D volume image based on a user-selected point in the ultrasound image data of the fibroid to be segmented. In various embodiments, the fibroid segmentation processor 160 may be provided as a deep neural network that may be made up of, for example, an input layer, an output layer, and one or more hidden layers in between the input and output layers. Each of the layers may be made up of a plurality of processing nodes that may be referred to as neurons. For example, the fibroid segmentation processor 160 may include an input layer having a neuron for each pixel, voxel, group of pixels, or group of voxels from the ultrasound image data. The output layer may have neurons corresponding to segmented fibroid(s). Each neuron of each layer may perform a processing function and pass the processed ultrasound image information to one of a plurality of neurons of a downstream layer for further processing. As an example, neurons of a first layer may learn to recognize edges of structure having the selected seed point in the ultrasound image data. The neurons of a second layer may learn to recognize shapes based on the detected edges from the first layer. The neurons of a third layer may learn positions of the recognized shapes relative to landmarks in the ultrasound image data. The processing performed by the fibroid segmentation processor 160 deep neural network (e.g., convolutional neural network) may segment fibroids depicted in ultrasound image data based on the user-provided seed points with a high degree of probability. The fibroid segmentation processor 160 may provide the ultrasound volume having the segmented uterus, endometrium, and fibroids to the rendering processor 180 for presenting ultrasound images and a volume rendering at the display system 134 and/or may store the ultrasound volume having the segmented fibroids, uterus, and endometrium at archive 138 and/or any suitable data storage medium.

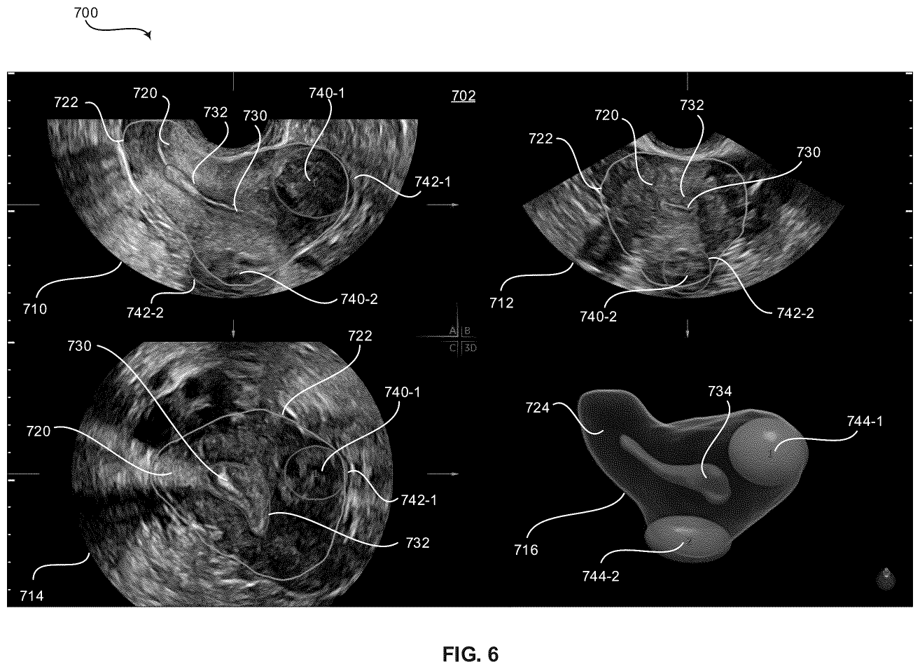

FIG. 6 is a screenshot 700 of an exemplary display 702 comprising an A-plane 710, B-plane 712, C-plane 714, and volume rendering 716 of a uterus 720, 722, 724, endometrium 730, 732, 734, and fibroids 740, 742, 744 segmented in an ultrasound volume, in accordance with various embodiments. Referring to FIG. 6, the screenshot 700 shows a display 702 having an A-plane 710. B-plane 712, C-plane 714, and volume rendering 716. The A-plane 710 may correspond with the mid-sagittal plane of the volume ultrasound image of the uterus 720. The B-plane 712 may correspond with the transverse plane of the volume ultrasound image of the uterus 720. The C-plane 714 may correspond with the coronal plane of the volume ultrasound image of the uterus 720. The volume rendering 716 is a two-dimensional (2D) projection of the three-dimensional (3D) ultrasound volume and comprises a rendering of the uterus 724, endometrium 734, a first fibroid 744-1, and a second fibroid 744-2. The A-plane 710, B-plane 712, and C-plane 714 are ultrasound image slices extracted from the acquired 3D ultrasound volume. The A-plane 710, B-plane 712, and C-plane 714 depict the uterus 720, endometrium 730, and fibroids 740. The A-plane 710, B-plane 712, and C-plane 714 may comprise markings 722, 732, 742-1, 742-2, colorized pixels, or the like identifying the segmentation of the uterus 722, the segmentation of the endometrium 732, the segmentation of the first fibroid 742-1, and the segmentation of the second fibroid 742-2. In various embodiments, the 3D ultrasound volume image comprises a plurality of parallel A-plane ultrasound image slices 710 spanning from one end of the 3D ultrasound volume to the opposite end of the 3D ultrasound volume. The ultrasound operator or other medical professional may operate the user input device 130 to scroll through the parallel A-plane ultrasound image slices 710 of the 3D ultrasound volume. For example, the ultrasound operator or other medical professional may operate the user input device 130 to scroll through and select seed points of fibroids 740-1, 740-2 for semi-automatic segmentation by the fibroid segmentation processor 160 using the user-provided seed points. As another example, the ultrasound operator or other medical professional may operate the user input device 130 to scroll through and manually draw the contours of the fibroids 740-1, 740-2 for manual segmentation.

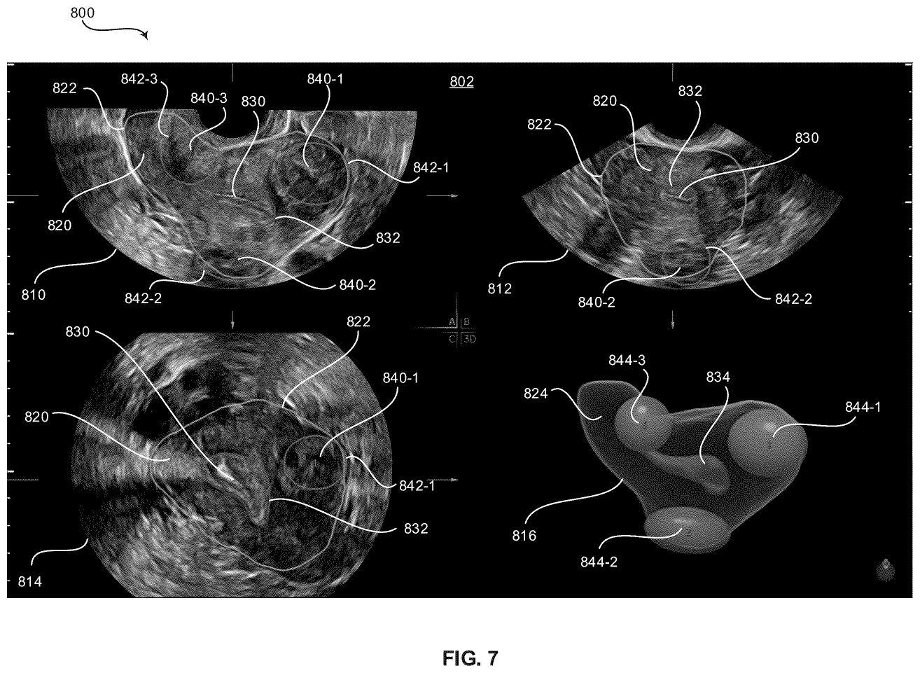

FIG. 7 is a screenshot 800 of an exemplary display 802 updated from the exemplary display 702 of FIG. 6 in response to a parallel shift operation to scroll between ultrasound image slices 710, 810 extending from a first end to a second end of an ultrasound volume, the exemplary updated display 802 comprising an A-plane 810, B-plane 812. C-plane 814, and volume rendering 816 of a uterus 820, 822, 824, endometrium 830, 832, 834, and fibroids 840, 842, 844 segmented in an ultrasound volume, in accordance with various embodiments. Referring to FIG. 7, the screenshot 800 shows a display 802 having an A-plane 810, B-plane 812, C-plane 814, and volume rendering 816. The A-plane 810, B-plane 812, C-plane 814, and volume rendering 816 share various characteristics with the A-plane 610, 710, B-plane 612, 712, C-plane 614, 714, and volume rendering 616, 716 of FIGS. 5 and 6 as described above. The A-plane 810, B-plane 812, and C-plane 814 may comprise markings 822, 832, 842-1, 842-2, 842-3, colorized pixels, or the like identifying the segmentation of the uterus 822, the segmentation of the endometrium 832, the segmentation of the first fibroid 842-1, the segmentation of the second fibroid 842-2, and the segmentation of a third fibroid 842-3. In various embodiments, the 3D ultrasound volume image comprises a plurality of parallel A-plane ultrasound image slices 810 spanning from one end of the 3D ultrasound volume to the opposite end of the 3D ultrasound volume. The ultrasound operator or other medical professional may operate the user input device 130 to scroll through the parallel A-plane ultrasound image slices 810 of the 3D ultrasound volume. For example, the ultrasound operator or other medical professional may operate the user input device 130 to scroll through and select seed points of fibroids 840-1, 840-2, 840-3 for semi-automatic segmentation by the fibroid segmentation processor 160 using the user-provided seed points. As another example, the ultrasound operator or other medical professional may operate the user input device 130 to scroll through and manually draw the contours of the fibroids 840-1, 840-2, 840-3 for manual segmentation.

The signal processor 132 may include a fibroid classification processor 170 that comprises suitable logic, circuitry, interfaces and/or code that may be operable to classify segmented fibroids 742-1, 742-2, 842-1, 842-2, 842-3 (collectively referred to as 742, 842). For example, the fibroid classification processor 170 may be configured to prompt an ultrasound operator or other medical professional to select a classification of each segmented fibroid 742, 842. As another example, the fibroid classification processor 170 may be configured to automatically classify each segmented fibroid 742, 842. In various embodiments, the classification may be the International Federation of Gynecology and Obstetrics (FIGO) uterine leiomyoma classification system, which assigns a classification based on the location of the fibroid 740, 742, 840, 842 with respect to the uterus 620, 622, 720, 722, 820, 822 and endometrium 630, 632, 730, 732, 830, 832. However, any suitable fibroid classification system may be applied, such as classifying the fibroids with respect to the patient (e.g., left/right, anterior/posterior, etc.). The FIGO uterine leiomyoma classification system is organized according to the location of the fibroid 740, 742, 840, 842 between the submucosal (0-2), intramural (3-5), and subserosal (6-7) layers of the uterus. A classification of “0” corresponds with pedunculated intracavity. A classification of “1” corresponds with less than 50 percent intramural. A classification of “2” corresponds with greater or equal to 50 percent intramural. A classification of “3” corresponds with 100 percent intramural and endometrial contact. A classification of “4” corresponds with 100 percent intramural. A classification of “5” corresponds with subserosal and greater than or equal to 50 percent intramural. A classification of “6” corresponds with subserosal and less than 50 percent intramural. A classification of “7” corresponds with subserosal pedunculated. A classification of “8” corresponds with a location of “other,” such as a cervical fibroid. In an exemplary embodiment, the fibroid classification processor 170 may present a pop up or any suitable prompt for receiving a selection of one of the classifications for each of the segmented fibroids 742, 842.

In certain embodiments, the fibroid classification processor 170 may include image analysis algorithms, artificial intelligence algorithms, one or more deep neural networks (e.g., a convolutional neural network) and/or may utilize any suitable form of image analysis techniques or machine learning processing functionality configured to automatically classify fibroids 740, 840 segmented 742, 842 from the ultrasound volume image and/or 2D ultrasound image slices 610, 612, 614, 710, 712, 714, 810, 812, 814 extracted from the 3D volume image based on a spatial location of each of the segmented fibroids 742, 842 with respect to the segmented uterus 622, 722, 822 and segmented endometrium 632, 732, 832. In various embodiments, the fibroid classification processor 170 may be provided as a deep neural network that may be made up of, for example, an input layer, an output layer, and one or more hidden layers in between the input and output layers. Each of the layers may be made up of a plurality of processing nodes that may be referred to as neurons. For example, the fibroid classification processor 170 may include an input layer having a neuron for each pixel, voxel, group of pixels, or group of voxels from the ultrasound image data. The output layer may have neurons corresponding to a classification of each segmented fibroid 742, 842. Each neuron of each layer may perform a processing function and pass the processed ultrasound image information to one of a plurality of neurons of a downstream layer for further processing. The processing performed by the fibroid classification processor 170 deep neural network (e.g., convolutional neural network) may classify fibroids 740-1, 740-2, 840-1, 840-2, 840-3 (collectively, 740, 840) depicted in ultrasound image data with a high degree of probability. The fibroid classification processor 170 may present the classifications of the fibroids 740, 840 at the display system 134 and/or may store the classifications of the fibroids 740, 840 at archive 138 and/or any suitable data storage medium.

The signal processor 132 may include a measurement processor 190 that comprises suitable logic, circuitry, interfaces and/or code that may be operable to perform measurements on the segmented uterus 622, 722, 822, endometrium 632, 732, 832, and fibroids 742, 842. For example, the measurement processor 190 may be configured to perform volume measurements, estimated diameter measurements, length measurements, width measurements, and/or any suitable measurements on the segmented anatomical structures. For purposes of the present application, the estimated diameter is defined as a maximum distance between points of the border of the segmented anatomical structure 622, 632, 722, 732, 742, 822, 832, 842. The measurement processor 190 may be configured to cause the display system 134 to present the measurement(s). The measurement processor 190 may be configured to store the measurement(s) at archive 138 and/or any suitable data storage medium.

In various embodiments, the signal processor 132 may be configured to receive a probe orientation of the ultrasound probe 104. For example, ultrasound probes 104 may include an orientation indicator, such as a knob. The signal processor 132 may be configured to receive a probe and/or acquisition type (e.g., transvaginal, transabdominal, and the like), an ultrasound probe direction (e.g., angle in degrees of the knob) and tilt (if applicable) from the ultrasound operator via the user input device 130. The probe orientation information may be applied by the signal processor 132 to present labels (e.g., left, right, anterior, posterior, and the like) to the ultrasound image views (e.g., A-plane 610, 710, 810, B-plane 612, 712, 812, C-plane 614, 714, 814, and volume rendering 616, 716, 816) presented at the display system 134 to provide spatial contextual information to an ultrasound operator.

In a representative embodiment, he signal processor 132 may be configured to generate a report comprising the ultrasound image data 610, 612, 614, 710, 712, 714, 810, 812, 814, volume rendering 616, 716, 816, fibroid classifications, measurements, and/or the like. The ultrasound image data 610, 612, 614, 710, 712, 714, 810, 812, 814 and volume renderings 616, 716, 816 included in the report provide exact locations of the fibroids 740, 840 with respect to uterus 620, 720, 820 and endometrium 630, 730, 830, which may be used for treatment planning, treatment monitoring, and clinical follow-ups. The generated report including the ultrasound image data 610, 612, 614, 710, 712, 714, 810, 812, 814, volume rendering 616, 716, 816, fibroid classifications, and/or measurements allow for longitudinal analysis with reference to subsequently acquired ultrasound image data 610, 612, 614, 710, 712, 714, 810, 812, 814, volume renderings 616, 716, 816, fibroid classifications, and measurements. For example, the signal processor 132 may be configured to present at the display system 134 comparisons illustrating relative changes in the fibroids 740, 840 with respect to the uterus 620, 720, 820 and endometrium 630, 730, 830.

Still referring to FIG. 1, the display system 134 may be any device capable of communicating visual information to a user. For example, a display system 134 may include a liquid crystal display, a light emitting diode display, and/or any suitable display or displays. The display system 134 can be operable to present the ultrasound images 610, 612, 614, 710, 712, 714, 810, 812, 814 extracted from the 3D ultrasound volume, the volume rendering(s) 616, 716, 816 of the segmented uterus 624, 724, 824, endometrium 634, 734, 834, and fibroid(s) 744-1, 744-2, 844-1, 844-2, 844-3 (collectively 744, 844), the fibroid classifications, measurements, reports, longitudinal analysis, probe orientation markers, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound images 610, 612, 614, 710, 712, 714, 810, 812, 814, segmented anatomical structures 622, 632, 722, 732, 742, 822, 832, 842, volume renderings 616, 716, 816 of the segmented anatomical structures 624, 634, 724, 734, 744, 824, 834, 844, fibroid classifications, measurements of the segmented anatomical structures 622, 632, 722, 732, 742, 822, 832, 842, generated reports related to the segmented anatomical structures 622, 632, 722, 732, 742, 822, 832, 842, instructions for segmenting anatomical structures 622, 632, 722, 732, 742, 822, 832, 842, instructions for generating renderings 624, 634, 724, 734, 744, 824, 834, 844 of the segmented anatomical structures 622, 632, 722, 732, 742, 822, 832, 842, instructions for classifying fibroids 740, 840, instructions for performing measurements on the segmented anatomical structures 622, 632, 722, 732, 742, 822, 832, 842, instructions for generating reports, instructions for presenting longitudinal analysis of the fibroids 740, 840 with respect to the uterus 620, 720, 820 and endometrium 630, 730, 830 over time, and/or any suitable images, information, and/or instructions, for example.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural networks (e.g., artificial intelligence model(s)) inferenced (i.e., deployed) by the uterus segmentation processor 140, endometrium segmentation processor 150, fibroid segmentation processor 160, and/or fibroid classification processor 170. For example, the segmentation or classification networks inferenced by the uterus segmentation processor 140, endometrium segmentation processor 150, fibroid segmentation processor 160, and/or fibroid classification processor 170 may be trained to automatically segment an anatomical structure (e.g., uterus 620, 720, 820, endometrium 630, 730, 830, fibroids 740, 840) or automatically classify a fibroid 740, 840 depicted in an ultrasound image. As an example, the training engine 210 may train the segmentation or classification network deployed by the uterus segmentation processor 140, endometrium segmentation processor 150, fibroid segmentation processor 160 and/or fibroid classification processor 170 to automatically segment a uterus 620, 720, 820, endometrium 630, 730, 830, and fibroids 740, 840 or automatically classify fibroids 740, 840 depicted in an ultrasound image 610, 612, 614, 710, 712, 714, 810, 812, 814 using database(s) 220 of classified anatomical structures (e.g., uterus 620, 720, 820, endometrium 630, 730, 830, and fibroids 740, 840). The classified anatomical structures may include an input image and a ground truth binary image (i.e., mask) of the manually segmented anatomical structure. The training engine 210 may be configured to optimize the segmentation or classification networks by adjusting the weighting of the segmentation or classification networks to minimize a loss function between the input ground truth mask and an output predicted mask.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms.

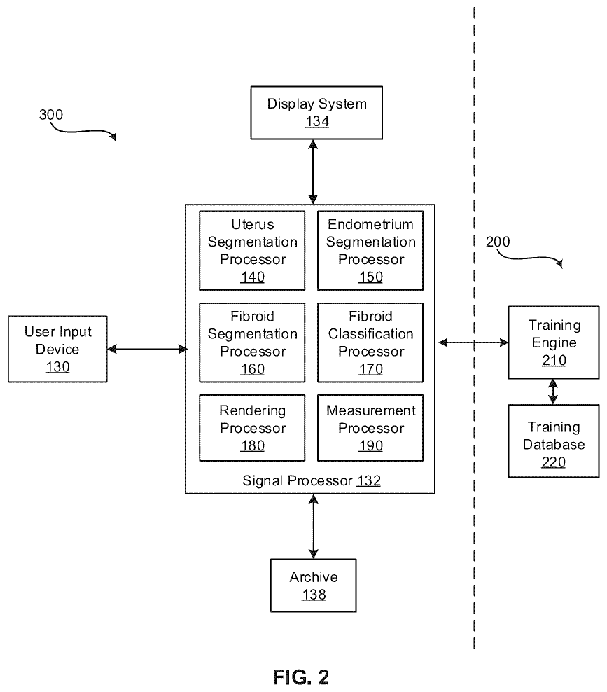

FIG. 2 is a block diagram of an exemplary medical workstation 300 that is operable to enhance visualization and documentation of fibroids 740, 840 with respect to a uterus 420, 520, 620, 720, 820 and endometrium 430, 530, 630, 730, 830 in ultrasound imaging, in accordance with various embodiments. In a representative embodiment, components of the medical workstation 300 and training system 200 may share various characteristics with components of the ultrasound system 100 and training system 200, as illustrated in FIG. 1 and described above. Referring to FIG. 2, the medical workstation 300 comprises a display system 134, a signal processor 132, an archive 138, and a user input device 130, among other things. Components of the medical workstation 300 may be implemented in software, hardware, firmware, and/or the like. The various components of the medical workstation 300 may be communicatively linked. Components of the medical workstation 300 may be implemented separately and/or integrated in various forms. For example, the display system 134 and the user input device 130 may be integrated as a touchscreen display.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as the ultrasound images 610, 612, 614, 710, 712, 714, 810, 812, 814 extracted from the 3D ultrasound volume, the volume rendering(s) 616, 716, 816 of the segmented uterus 624, 724, 824, endometrium 634, 734, 834, and fibroid(s) 744, 844, the fibroid classifications, measurements, reports, longitudinal analysis, probe orientation markers, and/or any suitable information.

The signal processor 132 may be one or more central processing units, microprocessors, microcontrollers, and/or the like. The signal processor 132 may be an integrated component, or may be distributed across various locations, for example. The signal processor 132 comprises a uterus segmentation processor 140, an endometrium segmentation processor 150, a fibroid segmentation processor 160, a fibroid classification processor 170, a rendering processor 180, and a measurement processor 190, as described above with reference to FIG. 1, and may be capable of receiving input information from a user input device 130 and/or archive 138, generating an output displayable by a display system 134, and manipulating the output in response to input information from a user input device 130, among other things. The signal processor 132, uterus segmentation processor 140, endometrium segmentation processor 150, fibroid segmentation processor 160, fibroid classification processor 170, rendering processor 180, and/or measurement processor 190 may be capable of executing any of the method(s) and/or set(s) of instructions discussed herein in accordance with the various embodiments, for example.

The archive 138 may be one or more computer-readable memories integrated with the medical workstation 300 and/or communicatively coupled (e.g., over a network) to the medical workstation 300, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound images 610, 612, 614, 710, 712, 714, 810, 812, 814, segmented anatomical structures 622, 632, 722, 732, 742, 822, 832, 842, volume renderings 616, 716, 816 of the segmented anatomical structures 624, 634, 724, 734, 744, 824, 834, 844, fibroid classifications, measurements of the segmented anatomical structures 622, 632, 722, 732, 742, 822, 832, 842, generated reports related to the segmented anatomical structures 622, 632, 722, 732, 742, 822, 832, 842, instructions for segmenting anatomical structures 622, 632, 722, 732, 742, 822, 832, 842, instructions for generating renderings 624, 634, 724, 734, 744, 824, 834, 844 of the segmented anatomical structures 622, 632, 722, 732, 742, 822, 832, 842, instructions for classifying fibroids 740, 840, instructions for performing measurements on the segmented anatomical structures 622, 632, 722, 732, 742, 822, 832, 842, instructions for generating reports, instructions for presenting longitudinal analysis of the fibroids 740, 840 with respect to the uterus 620, 720, 820 and endometrium 630, 730, 830 over time, and/or any suitable images, information, and/or instructions, for example.

The user input device 130 may include any device(s) capable of communicating information from a user and/or at the direction of the user to the signal processor 132 of the medical workstation 300, for example. As discussed above with respect to FIG. 1, the user input device 130 may include a touch panel, button(s), a mousing device, keyboard, rotary encoder, trackball, touch pad, camera, voice recognition, and/or any other device capable of receiving a user directive.

Still referring to FIG. 2, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the uterus segmentation processor 140, endometrium segmentation processor 150, fibroid segmentation processor 160, and/or fibroid classification processor 170. For example, the artificial intelligence model inferenced by the uterus segmentation processor 140, endometrium segmentation processor 150, fibroid segmentation processor 160, and/or fibroid classification processor 170 may be trained to automatically identify and/or classify a uterus 620, 720, 820, endometrium 630, 730, 830, fibroids 740, 840, or other anatomical objects of interest depicted in an ultrasound volume (or ultrasound slices 610, 612, 614, 710, 712, 714, 810, 812, 814 extracted from the ultrasound volume) using database(s) 220 of classified anatomical structures (e.g., uterus 620, 720, 820, endometrium 630, 730, 830, and fibroids 740, 840).

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the medical workstation 300 as shown in FIG. 2. Additionally and/or alternatively, components or all of the training system 200 may be integrated with the medical workstation 300 in various forms.

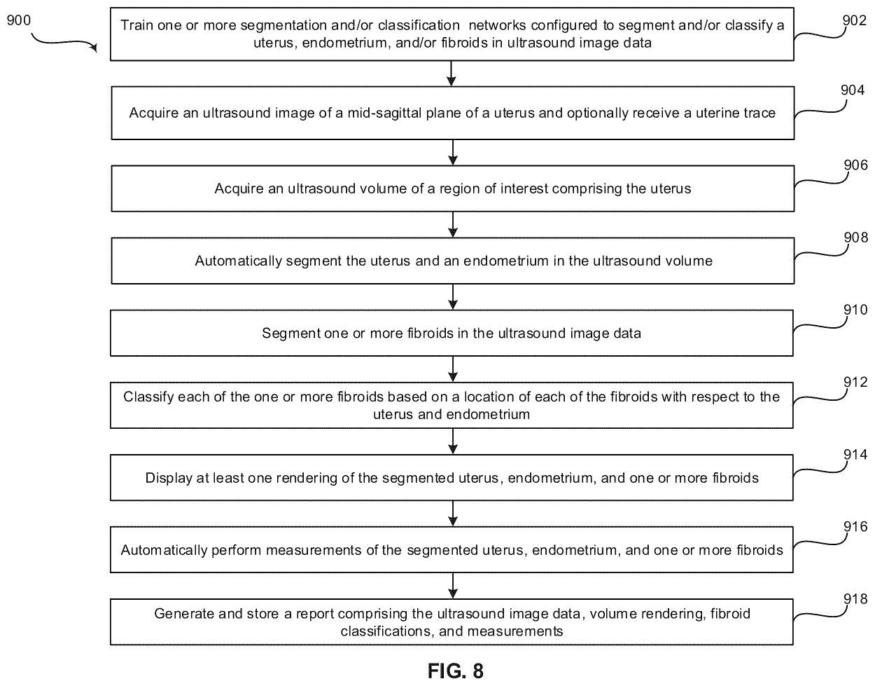

FIG. 8 is a flow chart 900 illustrating exemplary steps 902-918 that may be utilized for enhancing visualization and documentation of fibroids 740, 840 with respect to a uterus 620, 720, 820 and endometrium 630, 730, 830 in ultrasound imaging, in accordance with various embodiments. Referring to FIG. 8, there is shown a flow chart 900 comprising exemplary steps 902 through 918. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 902, one or more segmentation and/or classification networks are trained for segmenting and/or classifying a uterus 620, 720, 820, endometrium 630, 730, 830, and/or fibroids 740, 840 in ultrasound image data 610, 612, 614, 710, 712, 714, 810, 812, 814. For example, a training engine 210 of a training system 200 trains the segmentation and/or classification networks using database(s) 220 of classified anatomical structures (e.g., 620, 720, 820, endometrium 630, 730, 830, and fibroids 740, 840). The classified anatomical structures may include an input image and a ground truth binary image (i.e., mask) of the manually segmented anatomical structure. The training engine 210 may be configured to optimize the segmentation or classification networks by adjusting the weighting of the segmentation or classification networks to minimize a loss function between the input ground truth mask and an output predicted mask.

At step 904, an ultrasound probe 104 of an ultrasound system 100 acquires an ultrasound image of a mid-sagittal plane 410, 510 of a uterus 420, 520, and a signal processor 132 of the ultrasound system 100 optionally receives a uterine trace 536. For example, the ultrasound probe 104 may be navigated to identify a right endometrial plane (i.e., mid-sagittal plane of the uterus) 410, 510 via two-dimensional (2D) ultrasound scanning prior to initiating a three-dimensional (3D) ultrasound image acquisition at step 906. The acquired ultrasound image 410, 510 may be presented at a display system 134 of the ultrasound system 100. In various embodiments, the signal processor 132 may optionally receive, via the user input device 130, a uterine trace 536 through the endometrium 530 in the right endometrial plane (i.e., mid-sagittal plane) 510. The uterine trace 536 may be provided by the ultrasound operator via the user input device 130, such as a mousing device or a touchscreen display. The uterine trace 536 allows the ultrasound operator to confirm the mid-sagittal plane has been acquired prior to switching to the 3D image acquisition at step 906.

At step 906, an ultrasound probe 104 of an ultrasound system 100 acquires an ultrasound volume of a region of interest comprising the uterus 620, 720, 820. For example, the ultrasound probe 104 may perform a 3D acquisition to acquire an ultrasound volume aligned with the ultrasound probe position at the mid-sagittal plane 410, 510, 610, 710, 810. The acquired ultrasound volume may be provided to the rendering processor 180 to render various views (e.g., A-plane 610, 710, 810, B-plane 612, 712, 812, C-plane 614, 714, 814, volume rendering 616, 716, 816, etc.) for presentation at a display system 134 of the ultrasound system 100 and/or medical workstation 300, provided to a uterus segmentation processor 140, endometrium segmentation processor 150, fibroid segmentation processor 160, fibroid classification processor 170, and/or measurement processor 190 of the signal processor 132 of the ultrasound system 100 and/or medical workstation 300, and/or stored at archive 138 and/or any suitable data storage medium of the ultrasound system 100 and/or medical workstation 300.

At step 908, the signal processor 132 of the ultrasound system 100 and/or medical workstation 300 may automatically segment the uterus 620, 720, 820 and endometrium 630, 730, 830 in the ultrasound volume. For example, a uterus segmentation processor 140 of the signal processor 132 may be configured to deploy a segmentation network trained at step 902 to automatically segment the uterus 620, 720, 820 in the ultrasound volume received from the ultrasound probe 104 or retrieved from the archive 138 and/or any suitable data storage medium. As another example, an endometrium segmentation processor 150 of the signal processor 132 may be configured to deploy a segmentation network trained at step 902 to automatically segment the endometrium 630, 730, 830 in the ultrasound volume. The segmented ultrasound volume may be provided to the rendering processor 180 to render various views (e.g., A-plane 610, 710, 810, B-plane 612, 712, 812, C-plane 614, 714, 814, volume rendering 616, 716, 816, etc.) for presentation at a display system 134, provided to a fibroid segmentation processor 160, fibroid classification processor 170, and/or measurement processor 190 of the signal processor 132, and/or stored at archive 138 and/or any suitable data storage medium. In various embodiments, the signal processor 132 may be configured to receive a probe and/or acquisition type (e.g., transvaginal, transabdominal, and the like), an ultrasound probe direction (e.g., angle in degrees of the knob) and tilt (if applicable) from the ultrasound operator via the user input device 130. The probe orientation information may be applied by the signal processor 132 to present labels (e.g., left, right, anterior, posterior, and the like) to the ultrasound image views (e.g., A A-plane 610, 710, 810, B-plane 612, 712, 812, C-plane 614, 714, 814, volume rendering 616, 716, 816) presented at the display system 134 to provide spatial contextual information to an ultrasound operator.

At step 910, one or more fibroids 740, 840 may be segmented in the ultrasound image data 610, 612, 614, 710, 712, 714, 810, 812, 814. For example, the one or more fibroids 740, 840 may be segmented automatically, semi-automatically, and/or manually in the 3D ultrasound volume image and/or 2D ultrasound images slices 610, 612, 614, 710, 712, 714, 810, 812, 814 extracted from the 3D ultrasound volume image. For example, the fibroid segmentation processor 160 may be configured to receive manual traces provided by the ultrasound operator or other medical professional via a user input device 130. The ultrasound operator or other medical professional may scroll through the parallel A-plane ultrasound image slices 610, 710, 810 and manually trace the contours of identified fibroids 740, 840 using a mousing device, touchscreen, and/or any suitable user input device 130. As another example, the fibroid segmentation processor 160 may automatically segment fibroids 740, 840 from the 3D ultrasound volume image and/or 2D ultrasound images slices 610, 612, 614, 710, 712, 714, 810, 812, 814 extracted from the 3D ultrasound volume image with the assistance of color Doppler ultrasound data. As an example, the fibroid segmentation processor 160 may include image analysis algorithms, artificial intelligence algorithms, one or more deep neural networks (e.g., a convolutional neural network) and/or may utilize any suitable form of image analysis techniques or machine learning processing functionality configured to automatically segment fibroids 740, 840 from the ultrasound volume image and/or 2D ultrasound image slices 610, 612, 614, 710, 712, 714, 810, 812, 814 extracted from the 3D volume image using the color Doppler ultrasound image data. As another example, the fibroid segmentation processor 160 may additionally and/or alternatively be configured to semi-automatically segment fibroids 740, 840 from the 3D ultrasound volume image and/or 2D ultrasound images slices 610, 612, 614, 710, 712, 714, 810, 812, 814 extracted from the 3D ultrasound volume image with the assistance of user provided seed points identifying the fibroids 740, 840. For example, the fibroid segmentation processor 160 may include image analysis algorithms, artificial intelligence algorithms, one or more deep neural networks (e.g., a convolutional neural network) and/or may utilize any suitable form of image analysis techniques or machine learning processing functionality configured to semi-automatically segment fibroids 740, 840 from the ultrasound volume image and/or 2D ultrasound image slices 610, 612, 614, 710, 712, 714, 810, 812, 814 extracted from the 3D volume image based on a user-selected point in the ultrasound image data 610, 612, 614, 710, 712, 714, 810, 812, 814 of the fibroid 740, 840 to be segmented. The fibroid segmentation processor 160 may provide the ultrasound volume having the segmented uterus 622, 722, 822, endometrium 632, 732, 832, and fibroids 742, 842 to the rendering processor 180 for presenting ultrasound images 610, 612, 614, 710, 712, 714, 810, 812, 814 and a volume rendering 616, 716, 816 at the display system 134 and/or may store the ultrasound volume having the segmented uterus 622, 722, 822, endometrium 632, 732, 832, and fibroids 742, 842 at archive 138 and/or any suitable data storage medium.

At step 912, the signal processor 132 of the ultrasound system 100 and/or medical workstation 300 may classify each of the one or more fibroids 740, 840 based on a location of each of the fibroids 740, 840 with respect to the uterus 620, 720, 820 and endometrium 630, 730, 830. For example, a fibroid classification processor 170 of the signal processor 132 may be configured to prompt an ultrasound operator or other medical professional to select a classification of each segmented fibroid 742, 842. As another example, the fibroid classification processor 170 may be configured to automatically classify each segmented fibroid 742, 842. As an example, the fibroid classification processor 170 may include image analysis algorithms, artificial intelligence algorithms, one or more deep neural networks (e.g., a convolutional neural network) and/or may utilize any suitable form of image analysis techniques or machine learning processing functionality configured to automatically classify fibroids 740, 840 segmented from the ultrasound volume image and/or 2D ultrasound image slices 610, 612, 614, 710, 712, 714, 810, 812, 814 extracted from the 3D volume image based on a spatial location of each of the segmented fibroids 742, 842 with respect to the segmented uterus 622, 722, 822 and segmented endometrium 632, 732, 832. The fibroid classification processor 170 may present the classifications of the fibroids 740, 840 at the display system 134 and/or may store the classifications of the fibroids 740, 840 at archive 138 and/or any suitable data storage medium.

At step 914, the signal processor 132 of the ultrasound system 100 and/or medical workstation 300 may cause a display system 134 to present at least one rendering 610, 612, 614, 616, 710, 712, 714, 716, 810, 812, 814, 816 of the segmented uterus 622, 624, 722, 724, 822, 824, endometrium 632, 634, 732, 734, 832, 834, and one or more fibroids 742, 744, 842, 844. For example, a rendering processor 180 of the signal processor 132 may receive the ultrasound volume having the segmented uterus 622, 722, 822, endometrium 632, 732, 832, and/or fibroids 742, 842 from the uterus segmentation processor 140, endometrium segmentation processor 150, and/or fibroid segmentation processor 160, or may retrieve from archive 138 and/or any suitable data storage medium, the ultrasound volume having the segmented uterus 622, 722, 822, endometrium 632, 732, 832, and/or fibroids 742, 842. The rendering processor 180 may be configured to generate volume renderings 616, 716, 816, cross-sectional images, an A plane (i.e., the plane parallel to the acquisition plane) 610, 710, 810, a B plane (i.e., the plane perpendicular to the A plane but still parallel to the ultrasound beam) 612, 712, 812, a C plane (i.e., often referred to as the coronal plane, or the two-dimensional slices at various depths from and parallel to the transducer face, and perpendicular to the ultrasound beam) 614, 714, 814, curved slices (i.e., a 2D image slice extracted from a 3D volume along a curved anatomical structure, such as an endometrium of a uterus), and/or any suitable ultrasound images. The rendering processor 180 may be configured to cause a display system 134 to present the generated image(s) 610, 612, 614, 616, 710, 712, 714, 716, 810, 812, 814, 816 and/or store the generated image(s) 610, 612, 614, 616, 710, 712, 714, 716, 810, 812, 814, 816 at archive 138 and/or any suitable data storage medium.

At step 916, the signal processor 132 of the ultrasound system 100 and/or medical workstation 300 may automatically perform one or more measurements of the segmented uterus 622, 722, 822, endometrium 632, 732, 832, and/or one or more fibroids 742, 842. For example, a measurement processor 190 of the signal processor 132 that may be configured to perform measurements on the segmented uterus 622, 722, 822, endometrium 632, 732, 832, and fibroids 742, 842. As an example, the measurement processor 190 may be configured to perform volume measurements, estimated diameter measurements, length measurements, width measurements, and/or any suitable measurements on the segmented anatomical structures 622, 632, 722, 732, 742, 822, 832, 842. The measurement processor 190 may be configured to cause the display system 134 to present the measurement(s). The measurement processor 190 may be configured to store the measurement(s) at archive 138 and/or any suitable data storage medium.

At step 918, the signal processor 132 of the ultrasound system 100 and/or medical workstation 300 may generate and store a report comprising ultrasound image data 610, 612, 614, 710, 712, 714, 810, 812, 814, the volume rendering 616, 716, 816, the fibroid classifications, and the measurements. For example, the signal processor 132 may be configured to generate a report comprising the ultrasound image data 610, 612, 614, 710, 712, 714, 810, 812, 814, volume rendering 616, 716, 816, fibroid classifications, measurements, and/or the like. The ultrasound image data 610, 612, 614, 710, 712, 714, 810, 812, 814 and volume renderings 616, 716, 816 included in the report provide exact locations of the fibroids 740, 840 with respect to uterus 620, 720, 820 and endometrium 630, 730, 830, which may be used for treatment planning, treatment monitoring, and clinical follow-ups. The generated report including the ultrasound image data 610, 612, 614, 710, 712, 714, 810, 812, 814, volume rendering 616, 716, 816, fibroid classifications, and/or measurements allow for longitudinal analysis with reference to subsequently acquired ultrasound image data, volume renderings, fibroid classifications, and measurements. For example, the signal processor 132 may be configured to present at the display system 134 comparisons illustrating relative changes in the fibroids 740, 840 with respect to the uterus 620, 720, 820 and endometrium 630, 730, 830. The generated reports and longitudinal analysis may be presented at the display system 134 and/or stored at archive 138 and/or any suitable data storage medium.

Aspects of the present disclosure provide a method 900 and system 100, 300 for enhancing visualization and documentation of fibroid 740, 840 quantity, size, and location with respect to a uterus 620, 720, 820 and endometrium 630, 730, 830 in ultrasound imaging. In accordance with various embodiments, the method 900 may comprise receiving 906, by at least one processor 132, an ultrasound volume comprising ultrasound image data of a region of interest comprising a uterus 620, 720, 820. The method 900 may comprise automatically segmenting 908, by the at least one processor 132, 140, 150, the uterus 620, 720, 820 and an endometrium 630, 730, 830 in the ultrasound volume. The method 900 may comprise segmenting 910 one or more fibroids 740, 840 in the ultrasound image data. The method 900 may comprise generating a classification 912 of each of the one or more fibroids 740, 840 based on a location of each of the one or more fibroids 740, 840 with respect to the endometrium 630, 730, 830 and the uterus 620, 720, 820. The method 900 may comprise causing 914, by the at least one processor 132, 180, a display system 134 to present at least one image 610, 612, 614, 616, 710, 712, 714, 716, 810, 812, 814, 816 identifying the segmented uterus 622, 624, 722, 724, 822, 824, endometrium 632, 634, 732, 734, 832, 834, and one or more fibroids 742, 744, 842, 844.

In an exemplary embodiment, the method 900 may comprise acquiring 904 a two-dimensional (2D) ultrasound image of a mid-sagittal plane 410, 510 of the uterus 420, 520. The method 900 may comprise presenting the 2D ultrasound image 410, 510 at the display system 134. The ultrasound volume is acquired based on an alignment with the mid-sagittal plane 410, 510. The method 900 may comprise receiving 904 a uterine trace 536 of the endometrium 530 on the 2D ultrasound image of the mid-sagittal plane 510 of the uterus 520. In a representative embodiment, the segmenting 910 the one or more fibroids 740, 840 in the ultrasound image data 610, 612, 614, 710, 712, 714, 810, 812, 814 is performed semi-automatically by first individually presenting, at the display system 134, ultrasound image slices 610, 710, 810 of the ultrasound volume. The ultrasound image slices 610, 710, 810 are parallel and extend from a first end of an ultrasound volume to a second end of the ultrasound volume. Second, the at least one processor 132, 160 receives a seed point for each of one or more fibroids 740, 840 in the ultrasound image slices 610, 710, 810. Third, the at least one processor 132, 160 segments each of the one or more fibroids 740, 840 based on the corresponding seed point. In various embodiments, the segmenting 910 the one or more fibroids 740, 840 in the ultrasound image data is performed automatically by first receiving, by the at least one processor 132, 160, color Doppler ultrasound image data. Second, the at least one processor 132, 160 segments each of the one or more fibroids 740, 840 based on the color Doppler ultrasound image data. In certain embodiments, the segmenting 910 the one or more fibroids 740, 840 in the ultrasound image data is performed manually by receiving, by the at least one processor 132, 160, a manual trace of a contour of each of the one or more fibroids 740, 840. In an exemplary embodiment, the generating 912 the classification of each of the one or more fibroids 740, 840 comprises automatically classifying, by the at least one processor 132, 170, each of the one or more fibroids 740, 840 based on the location of each of the one or more fibroids 740, 840 with respect to the endometrium 630, 730, 830 and the uterus 620, 720, 820. In a representative embodiment, the generating 912 the classification of each of the one or more fibroids 740, 840 comprises presenting, for each of the one or more fibroids 740, 840, a prompt at the display system 134 and receiving a user input selecting the classification corresponding to the location with respect to the endometrium 630, 730, 830 and the uterus 620, 720, 820. In various embodiments, the method 900 comprises generating 916, by the at least one processor 132, 190, measurement data comprising one or both of a volume measurement and an estimated diameter measurement of each of the one or more fibroids 740, 840, the uterus 620, 720, 820, and/or the endometrium 630, 730, 830. In certain embodiments, the method 900 comprises generating 918, by the at least one processor 132, a report comprising the at least one image 610, 612, 614, 616, 710, 712, 714, 716, 810, 812, 814, 816, the classification of each of the one or more fibroids, and the measurement data. In an exemplary embodiment, the method 900 comprises causing, by the at least one processor, the display system 134 to present a comparison illustrating relative changes in the one or more fibroids 740, 840 with respect to the uterus 620, 720, 820 and the endometrium 630, 730, 830 over time.

Various embodiments provide a system 100 for enhancing visualization and documentation of fibroid 740, 840 quantity, size, and location with respect to a uterus 620, 720, 820 and endometrium 630, 730, 830 in ultrasound imaging. The system 100 may comprise an ultrasound probe 104, at least one processor 132, 140, 150, 160, 170, 180, 190 and a display system 134. The ultrasound probe 104 may be operable to acquire a two-dimensional (2D) ultrasound image of a mid-sagittal plane 410, 510 of a uterus 420, 520. The ultrasound probe 104 may be operable to acquire an ultrasound volume comprising ultrasound image data of a region of interest comprising the uterus 620, 720, 820. The ultrasound volume is acquired based on an alignment with the mid-sagittal plane 410, 510. The at least one processor 132, 140, 150 is configured to automatically segment the uterus 620, 720, 820 and an endometrium 630, 730, 830 in the ultrasound volume. The at least one processor 132, 160 is configured to segment one or more fibroids 740, 840 in the ultrasound image data. The at least one processor 132, 170 is configured to generate a classification of each of the one or more fibroids 740, 840 based on a location of each of the one or more fibroids 740, 840 with respect to the endometrium 630, 730, 830 and the uterus 620, 720, 820. The display system 134 is configured to present at least one image 610, 612, 614, 616, 710, 712, 714, 716, 810, 812, 814, 816 identifying the segmented uterus 622, 624, 722, 724, 822, 824, endometrium 632, 634, 732, 734, 832, 834, and one or more fibroids 742, 744, 842, 844.

In a representative embodiment, the at least one processor 132, 160 is configured to segment the one or more fibroids 740, 840 in the ultrasound image data semi-automatically. The at least one processor 132, 180 is configured to cause the display system 134 to individually present ultrasound image slices 610, 710, 810 of the ultrasound volume. The ultrasound image slices 610, 710, 810 are parallel and extend from a first end of an ultrasound volume to a second end of the ultrasound volume. The at least one processor 132, 160 is configured to receive a seed point for each of one or more fibroids 740, 840 in the ultrasound image slices 610, 710, 810. The at least one processor 132, 160 is configured to segment each of the one or more fibroids 740, 840 based on the corresponding seed point. In various embodiments, the at least one processor 132, 160 is configured to segment the one or more fibroids 740, 840 in the ultrasound image data automatically. The at least one processor 132, 160 is configured to receive color Doppler ultrasound image data. The at least one processor 132, 160 is configured to segment each of the one or more fibroids 740, 840 based on the color Doppler ultrasound image data. In certain embodiments, the at least one processor 132, 160 is configured to segment the one or more fibroids 740, 840 in the ultrasound image data manually. The at least one processor 132, 160 is configured to receive a manual trace of a contour of each of the one or more fibroids 740, 840. In an exemplary embodiment, the at least one processor 132, 170 is configured to automatically generate the classification of each of the one or more fibroids 740, 840 based on the location of each of the one or more fibroids 740, 840 with respect to the endometrium 630, 730, 830 and the uterus 620, 720, 820. In a representative embodiment, the at least one processor 132, 170 is configured to receive, for each of the one or more fibroids 740, 840, a user input selecting the classification corresponding to the location with respect to the endometrium 630, 730, 830 and the uterus 620, 720, 820 in response to the at least one processor 132, 170 causing the display system 134 to present, for each of the one or more fibroids 740, 840, a prompt to select the classification. In various embodiments, the at least one processor 132, 190 is configured to generate measurement data comprising one or both of a volume measurement and an estimated diameter measurement of each of the one or more fibroids 740, 840, the uterus 620, 720, 820, and/or the endometrium 630, 730, 830. In certain embodiments, the at least one processor 132 is configured to generate a report comprising the at least one image 610, 612, 614, 616, 710, 712, 714, 716, 810, 812, 814, 816, the classification of each of the one or more fibroids 740, 840, and the measurement data. In an exemplary embodiment, the at least one processor 132 is configured to cause the display system 134 to present a comparison illustrating relative changes in the one or more fibroids 740, 840 with respect to the uterus 620, 720, 820 and the endometrium 630, 730, 830 over time.

Certain embodiments provide a system 300 for enhancing visualization and documentation of fibroid 740, 840 quantity, size, and location with respect to a uterus 620, 720, 820 and endometrium 630, 730, 830 in ultrasound imaging. The system 300 may comprise at least one processor 132, 140, 150, 160, 170, 180, 190 and a display system 134. The at least one processor 132, 140, 150, 160, 170, 180, 190 may be configured to receive an ultrasound volume comprising ultrasound image data of a region of interest comprising a uterus 620, 720, 820. The at least one processor 132, 140, 150 may be configured to automatically segment the uterus 620, 720, 820 and an endometrium 630, 730, 830 in the ultrasound volume. The at least one processor 132, 160 may be configured to segment one or more fibroids 740, 840 in the ultrasound image data. The at least one processor 132, 170 may be configured to generate a classification of each of the one or more fibroids 740, 840 based on a location of each of the one or more fibroids 740, 840 with respect to the endometrium 630, 730, 830 and the uterus 620, 720, 820. The display system 134 may be configured to present at least one image 610, 612, 614, 616, 710, 712, 714, 716, 810, 812, 814, 816 identifying the segmented uterus 622, 624, 722, 724, 822, 824, endometrium 632, 634, 732, 734, 832, 834, and one or more fibroids 742, 744, 842, 844.

In various embodiments, the at least one processor 132, 160 is configured to segment the one or more fibroids 740, 840 in the ultrasound image data 610, 612, 614, 710, 712, 714, 810, 812, 814 semi-automatically. The at least one processor 132, 180 is configured to cause the display system 134 to individually present ultrasound image slices 610, 710, 810 of the ultrasound volume. The ultrasound image slices 610, 710, 810 are parallel and extend from a first end of an ultrasound volume to a second end of the ultrasound volume. The at least one processor 132, 160 is configured to receive a seed point for each of one or more fibroids 740, 840 in the ultrasound image slices 610, 710, 810. The at least one processor 132, 160 is configured to segment each of the one or more fibroids 740, 840 based on the corresponding seed point. In certain embodiments, the at least one processor 132, 160 is configured to segment the one or more fibroids 740, 840 in the ultrasound image data 610, 612, 614, 710, 712, 714, 810, 812, 814 automatically. The at least one processor 132, 160 is configured to receive color Doppler ultrasound image data. The at least one processor 132, 160 is configured to segment each of the one or more fibroids 740, 840 based on the color Doppler ultrasound image data. In an exemplary embodiment, the at least one processor 132, 160 is configured to segment the one or more fibroids 740, 840 in the ultrasound image data 610, 612, 614, 710, 712, 714, 810, 812, 814 manually. The at least one processor 132, 160 is configured to receive a manual trace of a contour of each of the one or more fibroids 740, 840. In a representative embodiment, the at least one processor 132, 170 is configured to automatically generate the classification of each of the one or more fibroids 740, 840 based on the location of each of the one or more fibroids 740, 840 with respect to the endometrium 630, 730, 830 and the uterus 620, 720, 820. In various embodiments, the at least one processor 132, 170 is configured to receive, for each of the one or more fibroids 740, 840, a user input selecting the classification corresponding to the location with respect to the endometrium 630, 730, 830 and the uterus 620, 720, 820 in response to the at least one processor 132, 170 causing the display system 134 to present, for each of the one or more fibroids 740, 840, a prompt to select the classification. In certain embodiments, the at least one processor 132, 190 is configured to generate measurement data comprising one or both of a volume measurement and an estimated diameter measurement of each of the one or more fibroids 740, 840, the uterus 620, 720, 820, and/or the endometrium 630, 730, 830. The at least one processor 132 is configured to generate a report comprising the at least one image 610, 612, 614, 616, 710, 712, 714, 716, 810, 812, 814, 816, the classification of each of the one or more fibroids 740, 840, and the measurement data. In an exemplary embodiment, the at least one processor 132 is configured to cause the display system 134 to present a comparison illustrating relative changes in the one or more fibroids 740, 840 with respect to the uterus 620, 720, 820 and the endometrium 630, 730, 830 over time.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhancing visualization and documentation of fibroid quantity, size, and location with respect to a uterus and endometrium in ultrasound imaging.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
receiving, by at least one processor, an ultrasound volume comprising ultrasound image data of a region of interest comprising a uterus;
automatically segmenting, by the at least one processor, the uterus and an endometrium in the ultrasound volume;
segmenting one or more fibroids in the ultrasound image data;
generating a classification of each of the one or more fibroids based on a location of each of the one or more fibroids with respect to the endometrium and the uterus; and
causing, by the at least one processor, a display system to present at least one image identifying the segmented uterus, endometrium, and one or more fibroids.

2. The method of claim 1, comprising:
acquiring a two-dimensional (2D) ultrasound image of a mid-sagittal plane of the uterus;
presenting the 2D ultrasound image at the display system, wherein the ultrasound volume is acquired based on an alignment with the mid-sagittal plane; and
receiving a uterine trace of the endometrium on the 2D ultrasound image of the mid-sagittal plane of the uterus.

3. The method of claim 1, wherein the segmenting the one or more fibroids in the ultrasound image data is performed semi-automatically by:
individually presenting, at the display system, ultrasound image slices of the ultrasound volume, wherein the ultrasound image slices are parallel and extend from a first end of an ultrasound volume to a second end of the ultrasound volume;
receiving, by the at least one processor, a seed point for each of one or more fibroids in the ultrasound image slices; and
segmenting, by the at least one processor, each of the one or more fibroids based on the corresponding seed point.

4. The method of claim 1, wherein the segmenting the one or more fibroids in the ultrasound image data is performed one of:
automatically by:
receiving, by the at least one processor, color Doppler ultrasound image data; and
segmenting, by the at least one processor, each of the one or more fibroids based on the color Doppler ultrasound image data; or manually by:
receiving, by the at least one processor, a manual trace of a contour of each of the one or more fibroids.

5. The method of claim 1, wherein the generating the classification of each of the one or more fibroids comprises one of:
automatically classifying, by the at least one processor, each of the one or more fibroids based on the location of each of the one or more fibroids with respect to the endometrium and the uterus; or
presenting, for each of the one or more fibroids, a prompt at the display system and receiving a user input selecting the classification corresponding to the location with respect to the endometrium and the uterus.

6. The method of claim 1, comprising generating, by the at least one processor, measurement data comprising one or both of a volume measurement and an estimated diameter measurement of:
each of the one or more fibroids,
the uterus, and/or
the endometrium.

7. The method of claim 6, comprising generating, by the at least one processor, a report comprising the at least one image, the classification of each of the one or more fibroids, and the measurement data.

8. The method of claim 1, comprising causing, by the at least one processor, the display system to present a comparison illustrating relative changes in the one or more fibroids with respect to the uterus and the endometrium over time.

9. A system comprising:
an ultrasound probe operable to:
acquire a two-dimensional (2D) ultrasound image of a mid-sagittal plane of a uterus;
acquire an ultrasound volume comprising ultrasound image data of a region of interest comprising the uterus, wherein the ultrasound volume is acquired based on an alignment with the mid-sagittal plane;
at least one processor configured to:
automatically segment the uterus and an endometrium in the ultrasound volume;
segment one or more fibroids in the ultrasound image data; and
generate a classification of each of the one or more fibroids based on a location of each of the one or more fibroids with respect to the endometrium and the uterus; and
a display system configured to present at least one image identifying the segmented uterus, endometrium, and one or more fibroids.

10. The system of claim 9, wherein the at least one processor is configured to segment the one or more fibroids in the ultrasound image data semi-automatically, wherein the at least one processor is configured to:
cause the display system to individually present ultrasound image slices of the ultrasound volume, wherein the ultrasound image slices are parallel and extend from a first end of an ultrasound volume to a second end of the ultrasound volume;
receive a seed point for each of one or more fibroids in the ultrasound image slices; and
segment each of the one or more fibroids based on the corresponding seed point.

11. The system of claim 9, wherein the at least one processor is configured to segment the one or more fibroids in the ultrasound image data one of:
automatically, wherein the at least one processor is configured to:
receive color Doppler ultrasound image data; and
segment each of the one or more fibroids based on the color Doppler ultrasound image data; or
manually, wherein the at least one processor is configured to:
receive a manual trace of a contour of each of the one or more fibroids.

12. The system of claim 9, wherein the at least one processor is configured to one of:
automatically generate the classification of each of the one or more fibroids based on the location of each of the one or more fibroids with respect to the endometrium and the uterus; or
receive, for each of the one or more fibroids, a user input selecting the classification corresponding to the location with respect to the endometrium and the uterus in response to the at least one processor causing the display system to present, for each of the one or more fibroids, a prompt to select the classification.

13. The system of claim 9, wherein the at least one processor is configured to generate measurement data comprising one or both of a volume measurement and an estimated diameter measurement of:
each of the one or more fibroids,
the uterus, and/or
the endometrium.

14. The system of claim 13, wherein the at least one processor is configured to generate a report comprising the at least one image, the classification of each of the one or more fibroids, and the measurement data.

15. The system of claim 9, wherein the at least one processor is configured to cause the display system to present a comparison illustrating relative changes in the one or more fibroids with respect to the uterus and the endometrium over time.

16. A system comprising:
at least one processor configured to:
receive an ultrasound volume comprising ultrasound image data of a region of interest comprising a uterus;
automatically segment the uterus and an endometrium in the ultrasound volume;
segment one or more fibroids in the ultrasound image data; and
generate a classification of each of the one or more fibroids based on a location of each of the one or more fibroids with respect to the endometrium and the uterus; and
a display system configured to present at least one image identifying the segmented uterus, endometrium, and one or more fibroids.

17. The system of claim 16, wherein the at least one processor is configured to segment the one or more fibroids in the ultrasound image data one of:
semi-automatically, wherein the at least one processor is configured to:
cause the display system to individually present ultrasound image slices of the ultrasound volume, wherein the ultrasound image slices are parallel and extend from a first end of an ultrasound volume to a second end of the ultrasound volume;
receive a seed point for each of one or more fibroids in the ultrasound image slices; and
segment each of the one or more fibroids based on the corresponding seed point;
automatically, wherein the at least one processor is configured to:
receive color Doppler ultrasound image data; and
segment each of the one or more fibroids based on the color Doppler ultrasound image data; or
manually, wherein the at least one processor is configured to:
receive a manual trace of a contour of each of the one or more fibroids.

18. The system of claim 16, wherein the at least one processor is configured to one of:
automatically generate the classification of each of the one or more fibroids based on the location of each of the one or more fibroids with respect to the endometrium and the uterus; or
receive, for each of the one or more fibroids, a user input selecting the classification corresponding to the location with respect to the endometrium and the uterus in response to the at least one processor causing the display system to present, for each of the one or more fibroids, a prompt to select the classification.

19. The system of claim 16, wherein the at least one processor is configured to generate:
measurement data comprising one or both of a volume measurement and an estimated diameter measurement of:
each of the one or more fibroids,
the uterus, and/or
the endometrium; and
a report comprising the at least one image, the classification of each of the one or more fibroids, and the measurement data.

20. The system of claim 16, wherein the at least one processor is configured to cause the display system to present a comparison illustrating relative changes in the one or more fibroids with respect to the uterus and the endometrium over time.
